(19) United States
(12) Patent Application Publication
(10) Pub. No.: US 2013/0203143 A1
(43) Pub. Date: Aug. 8, 2013

(54) Methods and Systems for the Production of Hydrocarbon Products

(71) Applicants: Michael Anthony Schultz, Roselle, IL (US); James Obern, Roselle, IL (US)

(72) Inventors: Michael Anthony Schultz, Roselle, IL (US); James Obern, Roselle, IL (US)

(73) Assignee: Lanza Tech New Zealand Limited, Auckland (NZ)

(21) Appl. No.: 13/879,605

(22) PCT Filed: Oct. 28, 2011
(86) PCT No.: PCT/US2011/058211

§ 371 Date: Apr. 15, 2013

Related U.S. Application Data
(60) Provisional application No. 61/408,216, filed on Oct. 29, 2010.

(51) Int. Cl. C12P 5/00

(52) U.S. Cl. CPC C12P 5/00
USPC 435/166; 435/289.1

(57) ABSTRACT

Methods and systems for the production of hydrocarbon products, including providing a substrate comprising CO to a bioreactor containing a culture of one or more micro-organisms; and fermenting the culture in the bioreactor to produce one or more hydrocarbon products. The substrate comprising CO is derived from a CO2 reforming process.

### FIELD OF THE INVENTION

This invention relates generally to methods for producing products, particularly hydrocarbon products such as alcohols, by microbial fermentation. In particular, the invention relates to producing hydrocarbon products from industrial gases associated with CO₂ reforming processes.

### BACKGROUND OF THE INVENTION

Ethanol is rapidly becoming a major hydrogen-rich liquid transport fuel around the world. Worldwide consumption of ethanol in 2005 was an estimated 12.2 billion gallons. The global market for the fuel ethanol industry has also been predicted to continue to grow sharply in future, due to an increased interest in ethanol in Europe, Japan, the USA and several developing nations.

For example, in the USA, ethanol is used to produce E10, a 10% mixture of ethanol in gasoline. In E10 blends, the ethanol component acts as an oxygenating agent, improving the efficiency of combustion and reducing the production of air pollutants. In Brazil, ethanol satisfies approximately 30% of the transport fuel demand, as both an oxygenating agent blended in gasoline, and as a pure fuel in its own right. Also, in Europe, environmental concerns surrounding the consequences of Green House Gas (GHG) emissions have been the stimulus for the European Union (EU) to set member nations a mandated target for the consumption of sustainable transport fuels such as biomass derived ethanol.

The vast majority of fuel ethanol is produced via traditional yeast-based fermentation processes that use crop derived carbohydrates, such as sucrose extracted from sugarcane or starch extracted from grain crops, as the main carbon source. However, the cost of these carbohydrate feed stocks is influenced by their value as human food or animal feed, and the cultivation of starch or sucrose-producing crops for ethanol production is not economically sustainable in all geographies. Therefore, it is of interest to develop technologies to convert lower cost and/or more abundant carbon resources into fuel ethanol.

CO is a major, free, energy-rich by-product of the incomplete combustion of organic materials such as coal or oil and oil derived products. For example, the steel industry in Australia is reported to produce and release into the atmosphere over 500,000 tonnes of CO annually.

Catalytic processes may be used to convert gases consisting primarily of CO and/or CO and hydrogen (H₂) into a variety of fuels and chemicals. Micro-organisms may also be used to convert these gases into fuels and chemicals. These biological processes, although generally slower than chemical reactions, have several advantages over catalytic processes, including higher specificity, higher yields, lower energy costs and greater resistance to poisoning.

The ability of micro-organisms to grow on CO as a sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl CoA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. While using CO as the sole carbon source, all such organisms produce at least two of these end products.

Anaerobic bacteria, such as those from the genus Clostridium, have been demonstrated to produce ethanol from CO, CO₂ and H₂ via the acetyl CoA biochemical pathway. For example, various strains of Clostridium Ijungdahlii that produce ethanol from gases are described in WO 00/68407, EP 117309, US patent nos. 5,173,429, 5,593,886, and 6,368,819, WO 98/00558 and WO 02/08438. The bacterium Clostridium autoethanogenum sp is also known to produce ethanol from gases (Abrini et al., Archives of Microbiology 161, pp 345-351 (1994)).

Although processes for the fermentation of substrates containing CO and H₂ by microorganisms are known, the potential for scaling and integrating these processes into an industrial context has barely been explored. Petrochemical plants and oil refineries produce large quantities of CO and H₂ as by-products and the potential exists to use this “waste” gas to produce valuable products. Additionally, a significant proportion of the waste gases are currently sent to flare (burned), or alternatively used as a source of fuel, both of which produce the undesirable greenhouse gas CO₂. Accordingly, there exists the potential to make improvements to industrial processes by exploiting the waste gases and energy produced thereby for use in fermentation to produce desirable products while simultaneously reducing gaseous carbon emissions from industrial plants.

Hydrogen is predicted to become a major feedstock for use in hydrogen fuel cells which are being developed for use in technology ranging from cars to consumer electronics. Further, it may be used as a combustible fuel. Hydrogen is also required in refineries for a large number of hydrotreating and hydrocracking processes, to remove sulphur, nitrogen and other impurities from hydrotreater feed and to hydrocrack heavier gas oils to distillates. As hydrogen production is capital intensive, it is desirable to develop methods that increase hydrogen production and recovery efficiency, especially from low-purity streams. In the absence of hydrogen recovery, such streams end up in fuel gas or sent to flare and the high-value hydrogen component is effectively wasted.

Carbon dioxide (CO₂) is currently the most significant greenhouse gas arising from anthropogenic activities (Treacy and Ross. Prepr. Pap.Am. Chem. Soc., 49 (1), 126, 2004). There is considerable pressure on industry to reduce carbon (including CO2) emissions and efforts are underway to capture the carbon prior to emission. Economic incentives for reducing carbon emissions and emissions trading schemes have been established in several jurisdictions in an effort to incentivise industry to limit carbon emissions in order to counteract climate change.

An option which may aid in the reduction of CO₂ emissions is the fixation of CO₂ as a chemical. The advantage of CO₂ fixation over CO₂ disposal (for example by sequestration in the deep ocean), is that the production of chemicals with an economic value is possible. CO₂ reforming (sometimes referred to as “dry” reforming) uses CO₂ and methane (CH₄) to produce carbon monoxide and hydrogen gas as products in the following reaction:

CO₂+CH₄→2CO+2H₂

The product of this reaction is often referred to as synthesis gas and is an equimolar mixture of CO and H₂. Synthesis gas can be used to produce higher value products, most notably sulphur free diesel, via Fischer-Tropsch synthesis:

nCO+(2n+1)H₂→CₙH₍₂ₙ₊₂₎+nH₂O

and methanol:

CO+2H₂→CH₃OH

However, both of these reactions require H₂ to be added to the reactant synthesis gas feed in order to establish the correct reactant ratio. This hydrogen would usually be supplied by the steam reforming of CH₄:

CH₄+H₂O→3H₂+CO

CO₂ and CH₄ are both relatively stable compounds with low potential energies. As a result the dry reforming reaction is highly endothermic and so energy has to be provided in order to drive it in the forward direction. Similarly, the steam reforming of CH₄ is also an endothermic reaction. The most likely energy source to drive these reactions will be the combustion of natural gas and this process, in itself, produces CO₂.

It is an object of the present invention to provide a process that overcomes or ameliorates at least one of the disadvantages of the prior art, or at least to provide the public with a useful choice.

### SUMMARY OF THE INVENTION

According to a first aspect, the invention provides a method of producing a hydrocarbon product, the method including:

i) providing a substrate comprising CO and/or H₂ to a bioreactor containing a culture of one or more micro-organisms;

ii) fermenting the culture in the bioreactor to produce one or more hydrocarbon products;

wherein the substrate comprising CO and/or H₂ is received from a CO₂ reforming process, the process being generally defined by the equation: CO₂+CH₄→2CO+2H₂.

Preferably, the CO₂ reforming process further comprises the regeneration of a catalyst wherein the regeneration produces a substrate containing CO and/or H₂.

Preferably, the substrate received from the CO₂ reforming process is passed to a pressure swing adsorption module prior to or after being received by the bioreactor.

Preferably, a post fermentation gaseous substrate output from the bioreactor comprising any one or more of CO₂, CH₄, CO, N₂ or H₂ is received by a membrane module adapted to separate one or more gases from one or more other gases.

Preferably, H₂ and CO₂ are separated from said gaseous substrate output from the bioreactor by the membrane module and passed to a pressure swing adsorption module.

Preferably, a gaseous substrate output from the bioreactor or membrane module comprising H₂ is received by a pressure swing adsorption module.

Preferably, the pressure swing adsorption module is used to recover H₂ from the gaseous substrate output from the bioreactor or membrane module.

Preferably, a gaseous substrate output from the bioreactor, the membrane module, or the PSA module, which comprises any one or more of CO₂, CH₄, CO or H₂ is reused in a CO₂ reforming process.

Preferably, a gaseous substrate output from the membrane module comprising any one or more of CO, CH₄ and/or N₂ is reused in a CO₂ reforming process or purged.

Preferably, the hydrocarbon produced by the bioreactor is reused in a CO₂ reforming process.

Preferably, a proportion of the CH₄ used for the CO₂ reforming process is received from the gasification of a refinery feedstock such as coal or vacuum gas oil. More preferably, the CH₄ is a component of substitute natural gas (SNG).

Preferably, the gaseous substrate comprising CO and/or H₂ received by the bioreactor has a further component of syngas or SNG received from a source other than the CO₂ reforming process. Preferably the source other than the CO₂ reforming process is gasification of a refinery feedstock such as coal or vacuum gas oil, although the invention is not limited thereto.

Preferably, a hydrocarbon reactant is passed through a prereformer prior to being used in a CO₂ reforming process.

Preferably, the hydrocarbon reactant is a hydrocarbon produced by the bioreactor.

Preferably, the hydrocarbon product or the hydrocarbon reactant is ethanol or propanol or butanol.

Preferably, the hydrocarbon product or the hydrocarbon reactant is a diol, more preferably 2,3-butanediol.

Preferably, the 2,3-butanediol is used for gasoline blending.

Preferably, the hydrocarbon produced is butyrate, propionate, caproate, propylene, butadiene, iso-butylene, or ethylene.

Preferably the hydrocarbon produced is a component of gasoline (about 8 carbon), jet fuel (about 12 carbon) or diesel (about 12 carbon).

Preferably, biomass is collected from the bioreactor and undergoes anaerobic digestion to produce a biomass product, preferably methane.

Preferably, the biomass product is used as a reactant for the CO₂ reforming process.

Preferably, the biomass product is used to produce supplemental heat to drive one or more reactions defined herein.

According to a second aspect, there is provided a CO₂ reforming process generally defined by the equation:

CO₂+CH₄→2CO+2H₂

wherein the CO₂ and/or CH₄ and/or components for the production of CO₂ and/or CH₄ is received from a bioreactor containing a culture of one or more microorganisms adapted to produce one or more hydrocarbon products by fermentation of a gaseous substrate comprising CO and/or H₂.

Preferably, the CO₂ reforming process is for treating and/or providing a substrate comprising CO and/or H₂ for a bioreactor.

Preferably, the gaseous substrate comprising CO and/or H₂ received by the bioreactor is corex gas and preferably comprises any one or more of CO, H₂, CO₂, N₂ or CH₄.

For the avoidance of doubt, the output of the bioreactor may undergo one or more processing steps before contributing to the reforming process.

Other features of the method of the second aspect are analogous to those of the method of the first aspect.

According to a third aspect, the invention provides a system for the production of a hydrocarbon product comprising:

a bioreactor containing a culture of one or more micro-organisms adapted to produce the hydrocarbon product by fermentation of a CO and/or H₂ containing substrate, wherein said substrate is received from a CO₂ reforming module adapted to carry out a CO₂ reforming process generally defined by the equation:

CO₂+CH₄→2CO+2H.

Preferably, the CO₂ reforming module further comprises a regenerator adapted to regenerate a catalyst by combustion of carboniferous deposits on the catalyst.

Preferably, the system comprises a gasification module adapted to gasify a refinery feedstock to produce syngas which may be used as a component of the CO containing substrate that is received by the bioreactor.

Preferably, the syngas is received by a substitute natural gas (SNG) module adapted to convert the syngas to SNG. Preferably, the CO₂ reforming module is adapted to receive SNG for use in a CO₂ reforming process.

Preferably, the bioreactor is adapted to receive the CO and/or H₂ containing substrate from, or pass said substrate to, a PSA module.

Preferably, the system further comprises a membrane module adapted to receive a gaseous substrate comprising any one or more of CO₂, CH₄, CO, N₂ or H₂ from the bioreactor and separate one or more gases from one or more other gases. More preferably, the membrane module is adapted to separate H₂ and/or CO₂ from said gaseous substrate.

Preferably, a PSA module is adapted to receive a gaseous substrate from the bioreactor or the membrane module.

Preferably, the PSA module is adapted to recover H₂ from the gaseous substrate.

Preferably, a CO₂ reforming module is adapted to receive a gaseous substrate from a bioreactor, a membrane module or a PSA module, wherein the gaseous substrate comprises any one or more of CO₂, H₂, CO and/or CH₄.

Preferably, a CO₂ reforming module is adapted to receive a hydrocarbon produced by the bioreactor.

Preferably, a CO₂ reforming module is adapted to receive a hydrocarbon from a prereformer module.

Preferably, the prereformer is adapted to receive a hydrocarbon produced by the bioreactor.

Preferably, the hydrocarbon is ethanol or propanol or butanol.

Preferably, the hydrocarbon is a diol, more preferably 2,3-butanediol.

Preferably, the 2,3-butanediol is used for gasoline blending.

Preferably, the hydrocarbon produced is butyrate, propionate, caproate, propylene, butadiene, iso-butylene, or ethylene.

Preferably the hydrocarbon produced is gasoline (about 8 carbon), jet fuel (about 12 carbon) or diesel (about 12 carbon).

As will be appreciated, any one of the aforementioned hydrocarbon products may be directly or indirectly produced i.e., further processing modules may be used to arrive at desired products.

Preferably, a digestion module is adapted to receive biomass from the bioreactor and produce a biomass product, preferably methane.

Preferably, the CO₂ reforming module is adapted to receive the biomass product for use as a reactant for the CO₂ reforming process.

Preferably, the digestion module is adapted to produce supplemental heat to be supplied to one or more other modules defined herein.

According to a fourth aspect, the invention provides a CO₂ reforming module adapted to perform a process generally defined by the equation:

CO₂+CH₄→2CO+2H₂

wherein the CO₂ and/or CH₄ and/or components for the production thereof is received from a bioreactor adapted to produce one or more hydrocarbon products by microbial fermentation of a gaseous substrate comprising CO and/or H₂.

Preferably, the CO₂ reforming module is adapted to treat and/or provide a substrate comprising CO and/or H₂ to a bioreactor.

Preferably, the bioreactor is adapted to receive corex gas which preferably comprises any one or more of CO, H₂, CO₂, N₂ or CH₄.

Other features of the system of the fourth aspect are analogous to those of the system of the third aspect.

According to a fifth aspect, the invention provides a method of capturing carbon from a substrate comprising CO, the method including:
(a) providing the substrate comprising CO and/or H₂ to a bioreactor containing a culture of one or more micro-organisms;
(b) fermenting the culture in the bioreactor to produce one or more hydrocarbon products;

wherein the substrate comprising CO is received from a CO₂ reforming module adapted to carry out a CO₂ reforming process generally defined by the equation:

CO₂+CH₄→2CO+2H₂.

Preferably, the substrate comprising CO is received from a pressure swing adsorption unit.

Preferably, the substrate comprising CO further comprises H₂.

According to a sixth aspect, the invention provides a method of capturing carbon from a substrate comprising CO, and/or H₂, wherein:

the substrate comprising CO and/or H₂ is provided to a bioreactor containing a culture of one or more micro-organisms and is fermented therein to produce one or more hydrocarbon products; the method including:

providing one or more products and/or by-products and/or waste products of the bioreactor and/or derivatives thereof to a CO₂ reforming module adapted to carry out a CO₂ reforming process generally defined by the equation:

CO₂+CH₄→2CO+2H₂.

According to a seventh aspect, the invention provides a hydrocarbon product when produced by the method of the first or second or fifth or sixth aspect, or the system of the third or fourth aspect.

Preferably, the hydrocarbon product is an alcohol, acid or diol.

Preferably, the hydrocarbon produced is butyrate, propionate, caproate , propylene, butadiene, iso-butylene, or ethylene.

Preferably the hydrocarbon produced is a component of gasoline (about 8 carbon), jet fuel (about 12 carbon) or diesel (about 12 carbon).

According to an eighth aspect, the invention provides hydrogen produced by CO₂ reforming wherein the hydrogen is received from a bioreactor containing a culture of one or more micro-organisms.

It will be appreciated by one of skill in the art that the CO₂ reforming process generally defined by the equation:

CO₂+CH₄→2CO+2H₂

may include further steps or reactions that are performed prior to, after, or concurrently with the reaction above. Aspects of the invention defined herein apply equally to these further steps or reactions.

The invention also includes the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE FIGURES

These and other aspects of the present invention, which should be considered in all its novel aspects, will become apparent from the following description, which is given by way of example only, with reference to the accompanying figures where:

FIG. 1 shows an exemplary system and method according to one embodiment.

FIG. 2 shows an exemplary system and method according to one embodiment in which the modules of the system are integrated to provide improved efficiency and carbon capture.

FIG. 3 shows an exemplary system comprising a gasification system operatively coupled to a CO₂ reforming system.

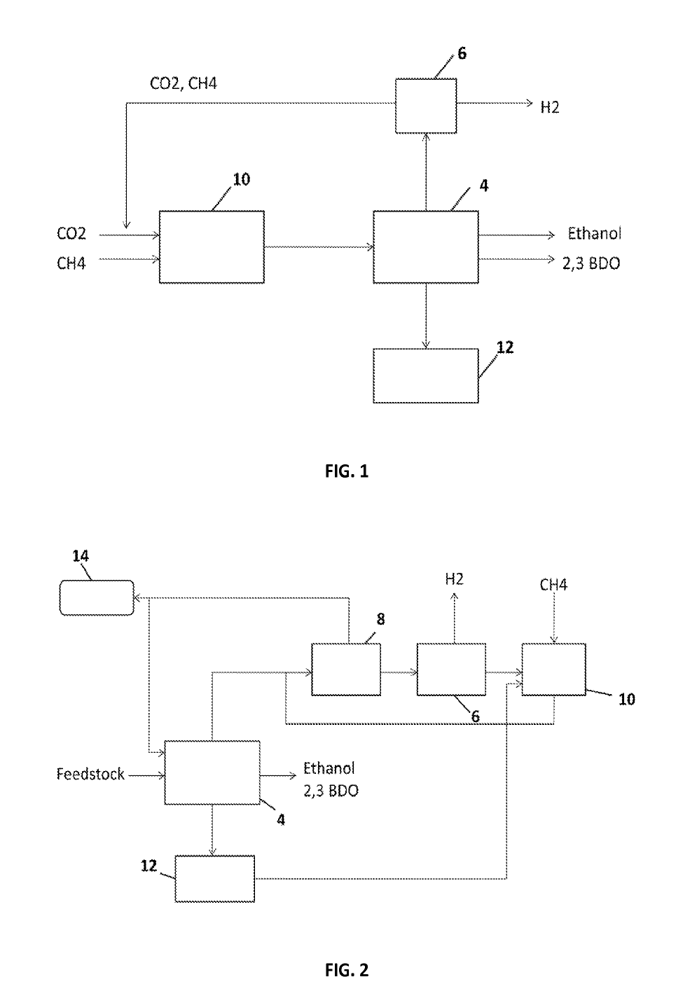

Note that the blocks of FIG. 1 represent both method steps and components of the physical system. Further, it will be appreciated that the arrangements shown are only preferred and that alternative ordering and combining of processing steps and modules are included within the scope of the invention.

### DETAILED DESCRIPTION OF THE INVENTION

Definitions

Unless otherwise defined, the following terms as used throughout this specification are defined as follows:

The term “substrate comprising carbon monoxide and/or hydrogen” and like terms should be understood to include any substrate in which carbon monoxide and/or hydrogen is available to one or more strains of bacteria for growth and/or fermentation, for example.

“Gaseous substrate comprising carbon monoxide and/or hydrogen” includes any gas which contains carbon monoxide and/or hydrogen. The gaseous substrate may contain a significant proportion of CO, preferably at least about 2% to about 100% CO by volume and/or preferably about 0% to about 95% hydrogen by volume.

In the context of fermentation products, the term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. The term “acetate” includes both acetate salt alone and a mixture of molecular or free acetic acid and acetate salt, such as the mixture of acetate salt and free acetic acid present in a fermentation broth as may be described herein. The ratio of molecular acetic acid to acetate in the fermentation broth is dependent upon the pH of the system.

The term “hydrocarbon” includes any compound that includes hydrogen and carbon. The term “hydrocarbon” incorporates pure hydrocarbons comprising hydrogen and carbon, as well as impure hydrocarbons and substituted hydrocarbons. Impure hydrocarbons contain carbon and hydrogen atoms bonded to other atoms. Substituted hydrocarbons are formed by replacing at least one hydrogen atom with an atom of another element. The term “hydrocarbon” as used herein includes compounds comprising hydrogen and carbon, and optionally one or more other atoms . The one or more other atoms include, but are not limited to, oxygen, nitrogen and sulfur. Compounds encompassed by the term “hydrocarbon” as used herein include at least acetate/acetic acid; ethanol, propanol, butanol, 2,3-butanediol, butyrate, propionate, caproate, propylene, butadiene, isobutylene, ethylene, gasoline, jet fuel or diesel.

The term “bioreactor” includes a fermentation device consisting of one or more vessels and/or towers or piping arrangements, which includes a Continuous Stirred Tank Reactor (CSTR), Immobilized Cell Reactor (ICR), Trickle Bed Reactor (TBR), Bubble Column, Gas Lift Fermenter, Membrane Reactor such as a Hollow Fibre Membrane Bioreactor (HFMBR), Static Mixer, or other vessel or other device suitable for gas-liquid contact.

Unless the context requires otherwise, the phrases “fermenting”, “fermentation process” or “fermentation reaction” and the like, as used herein, are intended to encompass both the growth phase and product biosynthesis phase of the process. In some embodiments the bioreactor may comprise a first growth reactor and a second fermentation reactor. As such, the addition of metals or compositions to a fermentation reaction should be understood to include addition to either or both of these reactors.

“Fermentation broth” is defined as the culture medium in which fermentation occurs.

“Refinery feedstock” is defined as a product or a combination of products derived from crude oil or coal and destined for further processing other than blending in the refining industry. It is transformed into one or more components and/or finished products and may include coal, heavy fuel oil, vacuum gas oil and heavy residual feedstock.

“Heavy residual feedstock” is defined as a very high boiling point portion of a petroleum crude oil, often generated as the heaviest fraction from a crude oil distillation system.

“Refinery process” includes any process normally carried out in an oil refinery or similar industrial context, including, but not limited to, fluid catalytic cracking, continuous catalytic regeneration reforming, gasification, CO₂ reforming, steam reforming and pressure swing adsorption.

The CO₂ Reforming Process

The CO₂ reforming process uses CO₂ and a hydrocarbon reactant (primarily methane from natural gas) and is generally defined by the equation:

CO₂+CH₄→2CO+2H₂.

Where methane is referred to herein, it will be appreciated by one of skill in the art that in alternative embodiments of the invention, the CO₂ reforming process may use other suitable hydrocarbon reactants, such as ethanol, methanol, propane, gasoline, autogas and diesel fuel, all of which may have differing reactant ratios and optimal conditions.

In a typical CO₂ reforming process, methane is reacted with CO₂ in a molar ratio of methane:CO₂ 1:1 at a pressure of 1 to 20 atm and temperature of approximately 900-1100° C. in the presence of a catalyst. Suitable catalysts are known in the art.

Conventionally the CO₂ reforming reactor is a packed bed reactor, in which the gas feeds are passed over a fixed bed of catalyst particles. Because the CO₂ reforming reaction produces carbon deposits that can interfere with the catalyst activity, alternate reactor systems may be used to mitigate this behaviour. For instance, a fluid bed reactor system is well known in the refining and petrochemical industries. Catalyst particles are fluidized using a gas feed stream, which may be composed of reactive species as well as inert species. The catalyst is transferred to a regenerator in which a gas stream containing oxygen, such as air, is used to combust the carbon deposits. The combustion results in production of a gaseous substrate containing varying proportions of CO and/or H₂ and may be suitable to be passed to a bioreactor for gas fermentation to produce a hydrocarbon product. The regenerated catalyst is returned to the reactor. The catalyst regeneration step also provides a way of transferring heat to the reactor system, as the exothermic reactions associated with carbon combustion produces heat. The catalyst particles serve as a medium to transfer this heat to the reactor system, which is useful for the endothermic CO₂ reforming reaction. Alternatively, the reactor system could be composed of multiple packed bed reactors, in which at any given time one or more reactors is fed with a gas containing methane and CO₂, at conditions suitable for the CO₂ reforming reaction, while one or more reactor systems is fed with an oxygen containing gas to combust the carbon deposited on the catalyst particles.

The CO₂ reforming process is typically followed by a Pressure Swing Adsorption (PSA) step to recover the purified hydrogen stream. The gas stream from the CO₂ reforming process enters a molecular sieve system which adsorbs CO₂, CO and CH₄ at high pressure. Hydrogen is able to pass through the sieve and is recovered for use in other applications. Once saturated, the sieve is depressurised then the desorbed gases are swept out using the smallest possible quantity of hydrogen product. The extent of regeneration is a function of pressure, as a greater quantity of adsorbed species is released at lower regeneration pressures. This, in turn, leads to greater hydrogen recovery. Therefore, regeneration pressures of close to atmospheric pressure maximize hydrogen recovery. The vessel is then repressurised with hydrogen ready for the next period as adsorber. Commercial systems will typically have three or four vessels to give a smooth operation.

The product of the CO₂ reaction is often referred to as synthesis gas and is an equimolar mixture of CO and H₂. Synthesis gas can be used to produce higher value products, most notably sulphur free diesel, via Fischer-Tropsch synthesis:

nCO+(2n+1)H₂→CₙH₍₂ₙ₊₂₎+nH₂O

and methanol:

CO+2H₂→CH₃OH

However, both of these reactions require H₂ to be added to the reactant synthesis gas feed in order to establish the correct reactant ratio. This hydrogen would usually be supplied by the steam reforming of CH₄:

CH₄+H₂O→3H₂+CO

The present invention provides a method of reducing the CO content of the gas received from the CO₂ reforming process. Among the advantages of this is that the level of additional hydrogen required for production of sulphur-free diesel and methanol is reduced or eliminated. Secondly, the present invention provides for recovery of hydrogen from the gas received from the CO₂ reforming process which can be used as a fuel source, such as to provide energy for the CO₂ reforming reaction, or used as a chemical feedstock, such as is required in refineries for various treating processes. Thirdly, the present invention enables the conversion of the CO₂ byproduct of the fermentation process into CO and H2, thus improving the efficiency of the fermentation. Fourthly, the present invention enables the conversion of external sources of CO₂ into hydrocarbon products.

According to one embodiment, the present invention provides a bioreactor which receives a CO and/or H₂ containing substrate from the CO₂ reforming process. The bioreactor contains a culture of one or more microorganisms capable of fermenting the CO and/or H₂ containing substrate to produce a hydrocarbon product. Thus, steps of a CO₂ reforming process may be used to produce, or improve the composition of, a gaseous substrate for a fermentation process.

Preferably, the bioreactor is adapted to receive a CO and/or H₂ containing substrate and contains a culture of one or more microorganisms capable of fermenting the CO and/or H₂ containing substrate to produce a hydrocarbon product.

According to an alternative embodiment, the CO₂ reforming process may be improved by providing an output of a bioreactor to the CO₂ reforming process. Preferably, the output is a gas and may enhance efficiency of the process and/or desired total product capture (for example of carbon or H₂).

The invention provides an integrated system of modules and processes with improved efficiency and carbon capture. An exemplary system exhibiting this integration is shown in FIG. 2.

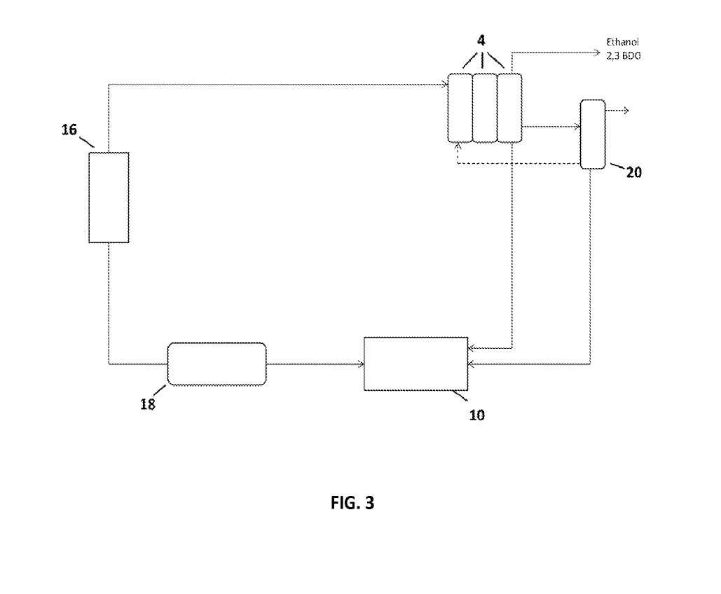

According to a further embodiment outlined in FIG. 3, the invention provides that a proportion of the CH₄ used for the CO₂ reforming process is received from the gasification of a refinery feedstock such as coal or vacuum gas oil. Gasification may be carried out according to processes known in the art. The gasification process involves the reaction of a refinery feedstock such as coal or vacuum gas oil with oxygen, preferably air, to produce syngas. The syngas may optionally be passed to a substitute natural gas (SNG) module which converts the syngas into SNG. SNG comprises primarily CH₄. The invention provides that SNG is used in addition to, or in place of, CH₄ from natural gas for the CO₂ reforming process. The syngas produced by the gasification process may also be fed to the bioreactor in combination with syngas produced from the CO₂ reforming process to produce a hydrocarbon product. Any CO or CO₂ vented from the bioreactor may be recycled for use in the CO₂ reforming process or another refinery process. The remaining SNG may be exported to the utility gas market or used in other refinery processes. Among the advantages of the above described embodiment is that the gasification process, the SNG production process, the CO₂ reforming process and the gas fermentation process are integrated with improved efficiency, carbon capture and hydrocarbon product formation when compared to known methods.

Preferably, the gaseous substrate comprising CO and/or H₂ received by the bioreactor has a further component of syngas or SNG received from a source other than the CO₂ reforming process. Preferably the source other than the CO₂ reforming process is gasification of a refinery feedstock such as coal or vacuum gas oil.

The Bioreactor

The fermentation may be carried out in any suitable bioreactor, such as a continuous stirred tank reactor (CSTR), an immobilised cell reactor, a gas-lift reactor, a bubble column reactor (BCR), a membrane reactor, such as a Hollow Fibre Membrane Bioreactor (HFMBR) or a trickle bed reactor (TBR). Also, in some embodiments of the invention, the bioreactor may comprise a first, growth reactor in which the micro-organisms are cultured, and a second, fermentation reactor, to which fermentation broth from the growth reactor may be fed and in which most of the fermentation product (e.g. ethanol and acetate) may be produced. The bioreactor of the present invention is adapted to receive a CO and/or H₂ containing substrate.

The CO₂ Reforming System

The bioreactor may be part of a system for the production of a hydrocarbon product wherein the system is generally as shown in FIG. 1 and comprises one or more modules selected from the group comprising:

a CO₂ reforming module adapted to produce CO and/or H₂ according to the CO₂ reforming process generally defined by the equation:

CO₂+CH₄→2CO+2H₂;

a pressure swing adsorption (PSA) module adapted to recover hydrogen from a gaseous substrate;

a membrane module adapted to separate one or more gases from one or more other gases, more preferably to separate H₂ and CO₂ from a gaseous substrate comprising any one or more of CO, H₂, CO₂, N₂ and CH₄;

a digestion module adapted to receive biomass from the bioreactor and produce a biomass product, preferably methane.

The PSA module may be adapted to receive a substrate from any one or more of the modules or the bioreactor. The PSA is adapted to recover hydrogen from the substrate. A post-fermentation substrate from the bioreactor may contain CO and/or H₂ and said substrate may be optionally recycled to the bioreactor to produce a hydrocarbon product. Alternatively, the hydrocarbon produced by the bioreactor may be used as a feedstock for the CO₂ reforming process.

The system may optionally include a prereformer module adapted to receive a hydrocarbon, which may be produced by the bioreactor. The prereformer is able to break down heavier hydrocarbons by a prereforming process to produce methane or other hydrocarbons suitable for the CO₂ reforming process.

It will be appreciated by one of skill in the art that the modules defined herein may be operatively coupled in any suitable arrangement to effect production of a desirable product.

The CO and/or H₂ containing substrate

The CO and/or H₂ containing substrate is captured or channelled from the process using any convenient method. Depending on the composition of the CO and/or H₂ containing substrate, it may also be desirable to treat it to remove any undesirable impurities, such as dust particles before introducing it to the fermentation. For example, the substrate may be filtered or scrubbed using known methods.

Typically, the CO will be added to the fermentation reaction in a gaseous state. However, methods of the invention are not limited to addition of the substrate in this state. For example, the carbon monoxide can be provided in a liquid. For example, a liquid may be saturated with a carbon monoxide containing gas and that liquid added to the bioreactor. This may be achieved using standard methodology. By way of example a microbubble dispersion generator (Hensirisak et. al. Scale-up of microbubble dispersion generator for aerobic fermentation; Applied Biochemistry and Biotechnology Volume 101, Number 3/October, 2002) could be used for this purpose. Where a “gas stream” is referred to herein, the term also encompasses other forms of transporting the gaseous components of that stream such as the saturated liquid method described above.

Gas Compositions

The CO-containing substrate may contain any proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 2%, may also be appropriate, particularly when H₂ and CO₂ are also present.

In a particular embodiment, the CO and/or H₂ containing substrate is corex gas. A typical corex gas composition comprises H₂ (16.1%), CO (43%), CO₂ (36.5%), N₂ (2.8%) and CH₄ (1.6%). The invention provides a method to convert the CO₂ and CH₄ in the corex gas to useful feed for the fermentation, thereby providing for additional utilization of the corex gas.

The presence of H₂ should not be detrimental to hydrocarbon product formation by fermentation. In particular embodiments, the presence of hydrogen results in an improved overall efficiency of alcohol production. For example, in particular embodiments, the substrate may comprise an approximate 2:1, or 1:1, or 1:2 ratio of H₂:CO. In other embodiments, the CO containing substrate comprises less than about 30% H₂, or less than 27% H₂, or less than 20% H₂, or less than 10% H₂, or lower concentrations of H₂, for example, less than 5%, or less than 4%, or less than 3%, or less than 2%, or less than 1%, or is substantially hydrogen free. In still other embodiments, the CO containing substrate comprises greater than 50% H₂, or greater than 60% H₂, or greater than 70% H₂, or greater than 80% H₂, or greater than 90% H₂.

The PSA step recovers hydrogen from the substrate received from the CO₂ reforming process, the membrane module or the bioreactor. In a typical embodiment, the substrate exiting the PSA step comprises about 10-35% H₂. The H₂ may pass through the bioreactor and be recovered from the substrate. In a particular embodiment of the invention, the H₂ is cycled to the PSA to be recovered from the substrate. The substrate may also contain some CO₂ for example, such as about 1% to about 80% CO₂ by volume, or 1% to about 30% CO₂ by volume.

Fermentation

Processes for the production of ethanol and other alcohols from gaseous substrates are known. Exemplary processes include those described for example in WO2007/117157, WO2008/115080, WO2009/022925, WO2009/064200, U.S. Pat. No. 6,340,581, U.S. Pat. No. 6,136,577, U.S. Pat. No. 5,593,886, U.S. Pat. No. 5,807,722 and U.S. Pat. No. 5,821,111, each of which is incorporated herein by reference.

Microorganisms

In various embodiments, the fermentation is carried out using a culture of one or more strains of carboxydotrophic bacteria. In various embodiments, the carboxydotrophic bacterium is selected from Moorella, Clostridium, Ruminococcus, Acetobacterium, Eubacterium, Butyribacterium, Oxobacter, Methanosarcina, Methanosarcina, and Desulfotomaculum. A number of anaerobic bacteria are known to be capable of carrying out the fermentation of CO to alcohols, including n-butanol and ethanol, and acetic acid, and are suitable for use in the process of the present invention. Examples of such bacteria that are suitable for use in the invention include those of the genus Clostridium, such as strains of Clostridium Ijungdahlii, including those described in WO 00/68407, EP 117309, U.S. Pat. Nos. 5,173,429, 5,593,886, and 6,368,819, WO 98/00558 and WO 02/08438, Clostridium carboxydivorans (Liou et al., International Journal of Systematic and Evolutionary Microbiology 33: pp 2085-2091), Clostridium ragsdalei (WO/2008/028055) and Clostridium autoethanogenum (Abrini et al, Archives of Microbiology 161: pp 345-351). Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). Further examples include Moorella thermoacetica, MooreIla thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp41-65). In addition, it should be understood that other acetogenic anaerobic bacteria may be applicable to the present invention as would be understood by a person of skill in the art. It will also be appreciated that the invention may be applied to a mixed culture of two or more bacteria.

One exemplary micro-organism suitable for use in the present invention is Clostridium autoethanogenum. In one embodiment, the Clostridium autoethanogenum is a Clostridium autoethanogenum having the identifying characteristics of the strain deposited at the German Resource Centre for Biological Material (DSMZ) under the identifying deposit number 19630. In another embodiment, the Clostridium autoethanogenum is a Clostridium autoethanogenum having the identifying characteristics of DSMZ deposit number DSMZ 10061. In another embodiment, the Clostridium autoethanogenum is a Clostridium autoethanogenum having the identifying characteristics of DSMZ deposit number DSMZ 23693. These strains have a particular tolerance to changes in substrate composition, particularly of H₂ and CO and as such are particularly well suited for use in combination with a CO₂ reforming process.

Culturing of the bacteria used in the methods of the invention may be conducted using any number of processes known in the art for culturing and fermenting substrates using anaerobic bacteria. By way of example, those processes generally described in the following articles using gaseous substrates for fermentation may be utilised: (i) K. T. Klasson, et al. (1991). Bioreactors for synthesis gas fermentations resources. Conservation and Recycling, 5; 145-165; (ii) K. T. Klasson, et al. (1991). Bioreactor design for synthesis gas fermentations. Fuel. 70. 605-614; (iii) K. T. Klasson, et al. (1992). Bioconversion of synthesis gas into liquid or gaseous fuels. Enzyme and Microbial Technology. 14; 602-608; (iv) J. L. Vega, et al. (1989). Study of Gaseous Substrate Fermentation: Carbon Monoxide Conversion to Acetate. 2. Continuous Culture. Biotech. Bioeng. 34. 6. 785-793; (v) J. L. Vega, et al. (1989). Study of gaseous substrate fermentations: Carbon monoxide conversion to acetate. 1. Batch culture. Biotechnology and Bioengineering. 34. 6. 774-784; (vi) J. L. Vega, et al. (1990). Design of Bioreactors for Coal Synthesis Gas Fermentations. Resources, Conservation and Recycling. 3. 149-160; all of which are incorporated herein by reference.

Fermentation Conditions

It will be appreciated that for growth of the bacteria and CO-to-hydrocarbon fermentation to occur, in addition to the CO-containing substrate, a suitable liquid nutrient medium will need to be fed to the bioreactor. A nutrient medium will contain vitamins and minerals sufficient to permit growth of the micro-organism used. Anaerobic media suitable for the production of hydrocarbon products through fermentation using CO as the sole carbon source are known in the art. For example, suitable media are described in U.S. Pat. Nos. 5,173,429 and 5,593,886 and WO 02/08438, WO2007/115157 and WO2008/115080 referred to above.

The fermentation should desirably be carried out under appropriate conditions for the desired fermentation to occur (e.g. CO-to-ethanol). Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition. Suitable conditions are described in WO02/08438, WO07/117157 and WO08/115080.

The optimum reaction conditions will depend partly on the particular micro-organism used. However, in general, it is preferred that the fermentation be performed at pressure higher than ambient pressure. Operating at increased pressures allows a significant increase in the rate of CO transfer from the gas phase to the liquid phase where it can be taken up by the micro-organism as a carbon source for the production of hydrocarbon products. This in turn means that the retention time (defined as the liquid volume in the bioreactor divided by the input gas flow rate) can be reduced when bioreactors are maintained at elevated pressure rather than atmospheric pressure. Also, since a given CO-to-hydrocarbon conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefits of conducting a gas-to-hydrocarbon fermentation at elevated pressures have also been described elsewhere. For example, WO 02/08438 describes gas-to-ethanol fermentations performed under pressures of 2.1 atm and 5.3 atm, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the hydrocarbon product is consumed by the culture.

Fermentation Products

Methods of the invention can be used to produce any of a variety of hydrocarbon products. This includes alcohols, acids and/or diols. More particularly, the invention may be applicable to fermentation to produce butyrate, propionate, caproate, ethanol, propanol, butanol, 2,3-butanediol, propylene, butadiene, iso-butylene and ethylene. These and other products may be of value for a host of other processes such as the production of plastics, pharmaceuticals and agrochemicals. In a particular embodiment, the fermentation product is used to produce gasoline range hydrocarbons (about 8 carbon), diesel hydrocarbons (about 12 carbon) or jet fuel hydrocarbons (about 12 carbon).

The invention also provides that at least a portion of a hydrocarbon product produced by the fermentation is reused in the CO₂ reforming process. In a particular embodiment, ethanol is cycled to be used as a feedstock for the CO₂ reforming process. In a further embodiment, the hydrocarbon feedstock and/or product is passed through a prereformer prior to being used in the CO₂ reforming process. Passing through a prereformer can increase the efficiency of hydrogen production and reduce the required capacity of the CO₂ reforming vessel.

The methods of the invention can also be applied to aerobic fermentations, to anaerobic or aerobic fermentations of other products, including but not limited to isopropanol. The methods of the invention can also be applied to aerobic fermentations, and to anaerobic or aerobic fermentations of other products, including but not limited to isopropanol.

Product Recovery

The products of the fermentation reaction can be recovered using known methods. Exemplary methods include those described in WO07/117157, WO08/115080, U.S. Pat. No. 6,340,581, U.S. Pat. No. 6,136,577, U.S. Pat. No. 5,593,886, U.S. Pat. No. 5,807,722 and U.S. Pat. No. 5,821,111. However, briefly and by way of example ethanol may be recovered from the fermentation broth by methods such as fractional distillation or evaporation, and extractive fermentation.

Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is also well known in the art.

Extractive fermentation procedures involve the use of a water-miscible solvent that presents a low toxicity risk to the fermentation organism, to recover the ethanol from the dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non volatile and is recovered for re-use in the fermentation.

Acetate, which may be produced as a by-product in the fermentation reaction, may also be recovered from the fermentation broth using methods known in the art.

For example, an adsorption system involving an activated charcoal filter may be used. In this case, it is preferred that microbial cells are first removed from the fermentation broth using a suitable separation unit. Numerous filtration-based methods of generating a cell free fermentation broth for product recovery are known in the art. The cell free ethanol—and Acetate—containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used. For example, U.S. Pat. Nos. 6,368,819 and 6,753,170 describe a solvent and cosolvent system that can be used for extraction of acetic acid from fermentation broths. As with the example of the oleyl alcohol-based system described for the extractive fermentation of ethanol, the systems described in U.S. Pat. Nos. 6,368,819 and 6,753,170 describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

The products of the fermentation reaction (for example ethanol and acetate) may be recovered from the fermentation broth by continuously removing a portion of the broth from the fermentation bioreactor, separating microbial cells from the broth (conveniently by filtration), and recovering one or more product from the broth simultaneously or sequentially.

In the case of ethanol it may be conveniently recovered by distillation, and acetate may be recovered by adsorption on activated charcoal, using the methods described above. The separated microbial cells are preferably returned to the fermentation bioreactor. The cell free permeate remaining after the ethanol and acetate have been removed is also preferably returned to the fermentation bioreactor. Additional nutrients (such as B vitamins) may be added to the cell free permeate to replenish the nutrient medium before it is returned to the bioreactor. Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

Biomass recovered from the bioreactor may undergo anaerobic digestion in a digestion module to produce a biomass product, preferably methane. This biomass product may be used as a feedstock for the CO₂ reforming process (optionally via a prereformer module) or used to produce supplemental heat to drive one or more of the reactions defined herein.

Gas Separation/Production

The fermentation of the present invention has the advantage that it is robust to the use of substrates with impurities and differing gas concentrations. Accordingly, production of a hydrocarbon product still occurs when a wide range of gas compositions is used as a fermentation substrate. The fermentation reaction may also be used as a method to separate and/or capture particular gases (for example CO) from the substrate and to concentrate gases, for example H₂, for subsequent recovery. When used in conjunction with one or more other processes as defined herein, the fermentation reaction may reduce the concentration of CO in the gas stream (substrate) and consequently concentrate H₂ which enables improved H₂ recovery.

The gas stream from the CO₂ reforming process may pass straight to the bioreactor for fermentation. Alternatively, the CO₂ reforming process may receive a gaseous substrate from the bioreactor, optionally via other processes. These differing arrangements could be advantageous by reducing costs and any energy loss associated with intermediate steps. Further, they may improve the fermentation process by providing a substrate having a higher CO content.

Since the composition of the gas stream is altered during its passage through the bioreactor, capture of components of the stream may be more efficiently performed after fermentation. Passing this stream to the CO₂ reforming step may thereby increase the efficiency of the CO₂ reforming process and/or the capture of one or more components of the stream. For instance, performing the PSA step after fermentation allows a higher regeneration pressure. While this will reduce the yield of hydrogen across the PSA step, the hydrogen can be recovered from at least a portion of the product of the fermentation. The higher regeneration pressure offers a less rigorous operating condition in the PSA step.

In a particular embodiment, the invention provides a membrane module adapted to receive a gaseous substrate from the bioreactor. Typically, the gaseous substrate from the bioreactor comprises CO, H₂, CO₂, N₂ or CH₄ and the membrane module is preferably adapted to separate one or more gases of the gaseous substrate. More preferably, the membrane module is adapted to separate H₂ and/or CO₂ from the gaseous substrate. This separation may
(a) improve the efficiency with which H₂ can be recovered from the substrate;
(b) allow the separated gases, preferably comprising CO, CH₄ and/or N₂ to be recycled to the bioreactor or purged from the system; and/or
(c) increase the purity of reactants to be passed to the CO₂ reforming module.

Trireforming

It is envisaged that the bioreactor of the present invention may also have utility when used in one or more reactions that are part of a trireforming process generally defined by the equations:

CH₄+CO₂→2CO+2H₂

CH₄+H₂O→CO+3H₂

CH₄+½O₂→CO+2H₂

CH₄+2O₂→CO₂+2H₂O

Carbon Capture

There is considerable pressure on industry to reduce carbon (including CO₂) emissions and efforts are underway to capture the carbon prior to emission. Economic incentives for reducing carbon emissions and emissions trading schemes have been established in several jurisdictions in an effort to incentivise industry to limit carbon emissions.

The present invention captures carbon from a substrate containing CO and/or H₂ and/Or CO₂ and/Or CH₄ via a fermentation process and produces a valuable hydrocarbon product (“valuable” is interpreted as being potentially useful for some purpose and not necessarily a monetary value). Typically, the CO produced by the CO₂ reforming process is converted to CO₂ by burning or by a water-gas shift reaction. The CO₂ reforming process and subsequent burning also typically results in release of CO₂ to the atmosphere. The invention provides a method of capturing the carbon that would otherwise be vented to the atmosphere as a hydrocarbon product. Where the energy produced is used to generate electricity, there are likely to be considerable losses in energy due to the transmission along high-voltage power lines. In contrast, the hydrocarbon product produced by the present invention may be easily transported and delivered in a usable form to industrial, commercial, residential and transportation end-users resulting in increased energy efficiency and convenience. The production of hydrocarbon products that are formed from what are effectively waste gases is an attractive proposition for industry. This is especially true for industries situated in remote locations if it is logistically feasible to transport the product long distances. Thus, the invention can provide for increased carbon capture as well as improve H₂ production.

General

Embodiments of the invention are described by way of example. However, it should be appreciated that particular steps or stages necessary in one embodiment may not be necessary in another. Conversely, steps or stages included in the description of a particular embodiment can be optionally advantageously utilised in embodiments where they are not specifically mentioned.

While the invention is broadly described with reference to any type of stream that may be moved through or around the system(s) by any known transfer means, in certain embodiments reformed and/or blended substrate streams are gaseous. Those skilled in the art will appreciate that particular stages may be coupled by suitable conduit means or the like, configurable to receive or pass streams throughout a system. A pump or compressor may be provided to facilitate delivery of the streams to particular stages. Furthermore, a compressor can be used to increase the pressure of gas provided to one or more stages, for example the bioreactor. As discussed hereinabove, the pressure of gases within a bioreactor can affect the efficiency of the fermentation reaction performed therein. Thus, the pressure can be adjusted to improve the efficiency of the fermentation. Suitable pressures for common reactions are known in the art.

In addition, the systems or processes of the invention may optionally include means for regulating and/or controlling other parameters to improve overall efficiency of the process. For example particular embodiments may include determining means to monitor the composition of substrate and/or exhaust stream(s). In addition, particular embodiments may include a means for controlling the delivery of substrate stream(s) to particular stages or elements within a particular system if the determining means determines the stream has a composition suitable for a particular stage. For example, in instances where a gaseous substrate stream contains low levels of CO or high levels of O₂ that may be detrimental to a fermentation reaction, the substrate stream may be diverted away from the bioreactor. In particular embodiments of the invention, the system includes means for monitoring and controlling the destination of a substrate stream and/or the flow rate, such that a stream with a desired or suitable composition can be delivered to a particular stage.

In addition, it may be necessary to heat or cool particular system components or substrate stream(s) prior to or during one or more stages in the process. In such instances, known heating or cooling means may be used.

Various embodiments of the systems of the invention are described in the accompanying Figures.

The alternative embodiments described in FIGS. 1 to 3 comprise features in common with one another and the same reference numbers have been used to denote the same or similar features in the various figures. Only the new features (relative to the preceding Figures) are described, and so the Figures should be considered in conjunction with the description of FIG. 1.

FIG. 1 shows a system for the production of a hydrocarbon in accordance with one embodiment of the invention. The system of FIG. 1 comprises:
a CO₂ reforming module 10 adapted to produce CO and/or H₂ according to the CO₂ reforming process generally defined by the equation:

CO₂+CH₄→2CO+2H₂;
a pressure swing adsorption (PSA) module 6 adapted to recover hydrogen from a gaseous substrate;
a membrane module (not shown) adapted to separate one or more gases from one or more other gases, more preferably to separate H₂ and CO₂ from a gaseous substrate comprising any one or more of CO, H₂, CO₂, N₂ and CH₄;
a digestion module 12 adapted to receive biomass from the bioreactor and produce a biomass product, preferably methane.

The PSA module 6 may be adapted to receive a substrate from any one or more of the modules or the bioreactor 4. The PSA 6 is adapted to recover hydrogen from the substrate. A post-fermentation substrate from the bioreactor 4 may contain CO and/or H₂ and said substrate may be optionally recycled to the bioreactor to produce a hydrocarbon product. Alternatively, the hydrocarbon produced by the bioreactor may be used as a feedstock for the CO₂ reforming process.

The system may optionally include a prereformer module adapted to receive a hydrocarbon, which may be produced by the bioreactor. The prereformer is able to break down heavier hydrocarbons by a prereforming process to produce methane or other hydrocarbons suitable for the CO₂ reforming process.

FIG. 2 depicts a method and system for the integration of a CO2 reforming system in accordance with one embodiment of the invention. With reference to FIG. 2 a substrate comprising CO and/or H₂ is passed into a bioreactor 4. The CO and/or H₂ substrate is fermented in the bioreactor to produce ethanol and/or 2,3 Butanediol (2,3 BDO). A gas stream exiting the bioreactor 4 is passed through a membrane 8, said membrane 8 being configured to to separate one or more gases from one or more other gases. Typically cases such as Ch₄ and N₂ are captured by the membrane 8 and purged 14. The remaining gas stream comprising CO and H₂ is then passed to the PSA module 6, wherein at least a portion of the hydrogen is recovered from the gas stream. The gas stream exiting the PSA module 6 is passed into the CO₂ reformer 10 wherein the gas stream is converted to a substrate comprising CO, which can then be passed back to the bioreactor 4. In certain embodiments of the invention, the substrate comprising CO and/or H₂ passed to the bioreactor is produced by a CO₂ reforming system.

FIG. 3 is an example of one embodiment of the invention, wherein the invention provides that a portion of the CH₄ used for the CO₂ reforming process is received from the gasification of a refinery feedstock. FIG. 3 shows a system for producing a hydrocarbon product, the system comprising a CO₂ reforming module and a bioreactor. The CO₂ reforming module comprises a gasification module 16, a substitute natural gas module 18, and a CO₂ reformer. The gasification module 16 configured to produce syngas from the gasification of a refinery feedstock such as coal or gas. Gasification may be carried out according to processes known in the art. The gasification module 16 comprises at least a gasification unit. The gasification module may also comprise additional features including heat exchange units and gas cleaning means. At least a portion of the syngas produced by the gasification module 16 is passed to a bioreactor module 4. A further portion of the syngas produced by the gasification module 16 is passed to a Substitute Natural Gas (SNG) module 18. The SNG module 18 comprises a substitute natural gas catalytic reactor configured to convert the syngas received from the gasification module 16 to SNG, said SNG comprising primarily methane (CH4). The SNG stream from the SNG module 18 is then passed to a CO₂ reformer 10 wherein it is reacted with CO₂ to produce a gaseous substrate comprising CO and H₂ according to the following stoichiometry; CO₂+CH₄→2CO+2H₂. The substrate comprising CO and H₂ is then passed to a gas separation module 20. The gas separation module 20 may comprise any known gas separation means. An exemplary gas separation means is a pressure swing adsorption means. As shown in FIG. 3, at least a portion of the hydrogen in the substrate stream is separated from the stream and recovered. The remaining CO rich gas stream is then passed to the bioreactor 4. In the bioreactor 4 containing a culture of one or more microorganism, the substrate comprising CO and/or H₂ is fermented to produce one or more hydrocarbon products. The hydrocarbon products in one embodiment are ethanol and 2,3-butanediol.In certain embodiments, a tail gas comprising CO₂ and H₂ exiting the bioreactor 4, is passed directly to the CO₂ reformer 10. In certain embodiments the tail gas exiting the bioreactor 4 is first passed to the gas separation module 20 wherein the H₂ is separated and recovered, and the remaining CO₂ rich gas stream is passed to the CO₂ reformer 10.

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

Where reference has been made in the foregoing description to integers having known equivalents thereof, those integers are herein incorporated as if individually set forth.

Furthermore, titles, heading, or the like are provided to enhance the reader's comprehension of this document, and should not be read as limiting the scope of the present invention. The entire disclosures of all applications, patents and publications cited above and below, if any, are herein incorporated by reference.

The reference to any prior art in this specification is not, and should not be taken as, an acknowledgement or any form of suggestion that that prior art forms part of the common general knowledge in the field of endeavour in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1-18. (canceled)

19. A method for producing a hydrocarbon product, the method comprising;
a) producing a gaseous substrate comprising CO and optionally H₂ in a CO₂ reforming process;
b) passing the gaseous substrate comprising CO and optionally H₂ to a bioreactor containing at least one microorganism;
c) fermenting the culture in the bioreactor to produce at least one hydrocarbon product, and a post fermentation exit gas stream comprising at least one gas selected from the group consisting of CO₂, CH₄, N₂ and H₂.

20. The method of claim 19 wherein the CO₂ reforming process comprises the regeneration of a catalyst to produce the substrate of (a).

21. The method of claim 20 wherein the post fermentation exit gas stream is received by a membrane module configured to separate one or more gases from one or more other gases.

22. The method of claim 21 wherein H₂ and CO₂ are separated as a gas stream from the post fermentation exit gas stream by the membrane module.

23. The method of claim 19 wherein the post fermentation exit gas stream from the bioreactor is passed to a gas separation unit operated at conditions to separate and recover hydrogen from the post fermentation exit gas stream.

24. The method of claim 22 wherein the H₂ and CO₂ gas stream is passed to a gas separation unit operated at conditions to separate and recover hydrogen from the H2 and CO2 gas stream.

25. The method of claim 23 where the gas separation unit is a pressure swing adsorption module.

26. The method of claim 24 where the gas separation unit is a pressure swing adsorption module.

27. The method of claim 19 wherein the at least one hydrocarbon product is selected from the group consisting of ethanol, propanol, butanol, 2,3-butanediol, acetate, butyrate, propionate, caproate, propylene, butadiene, isobutylene, ethylene, gasoline, jet fuel or diesel fuel.

28. The method of claim 27 where the at least one hydrocarbon product is ethanol, 2,3-butanediol or mixtures thereof.

29. A system for the production of a hydrocarbon product, the system comprising;
a) a bioreactor containing a culture of at least one microorganism adapted to produce the hydrocarbon product by fermentation of a CO or a CO and H₂ containing substrate, wherein the bioreactor is adapted to receive the CO or CO and H₂ containing substrate from a CO₂ reforming module;
b) a CO₂ reforming module comprising a CO₂ reformer configured to produce a gaseous substrate comprising CO or CO and H₂;
c) a gas separation unit;
d) means for supplying a substrate comprising CO or CO and H₂ from the CO₂ reforming module of (b) to the bioreactor (a); and
e) means for supplying a post fermentation exit gas stream from the bioreactor to the gas separation unit, the exit gas stream comprising at least one gas selected from the group consisting of CO₂, CH₄, N₂ and H₂.

30. The system of claim 29 wherein the CO₂ reforming module further comprises a regenerator adapted to regenerate a catalyst by combustion of carboniferous deposits on the catalyst.

31. The system of claim 29 wherein the CO₂ reforming module further comprises a gasification module adapted to gasify a refinery feedstock to produce a syngas stream.

32. The system of claim 31 wherein the CO₂ reforming module further comprises a substitute natural gas (SNG) module adapted to receive at least a portion of the syngas and convert the at least a portion of the syngas to SNG.

33. The system of claim 32 wherein the CO₂ reformer is configured to receive at least a portion of the substitute natural gas.

34. The system of claim 29 wherein the bioreactor is adapted to receive the substrate produced by the CO₂ reformer or the syngas produced by the gasification module.

35. The system of claim 29 wherein the gas separation unit is a membrane module or a pressure swing adsorption (PSA) module configured to separate CO or CO and H₂ from one or more other gases in a gas stream exiting the bioreactor.

36. The system of claim 29 wherein the CO₂ reforming module is adapted to receive a gaseous substrate from the bioreactor or the gas separation unit.
